(19) United States
(12) Patent Application Publication
(10) Pub. No.: US 2010/0022819 A1
(43) Pub. Date: Jan. 28, 2010

(54) Apparatus and methods for diffusion of aromatic substances in ventilatory equipment

(76) Inventor: Bret Floyd Randall, South Jordan, UT (US)

Correspondence Address: TRASKBRITT, P.C., P.O. BOX 2550, SALT LAKE CITY, UT, 84110, US

(21) Appl. No.: 12/311,308

(22) PCT Filed: Sep. 27, 2007
(86) PCT No.: PCT/US2007/020892

§ 371 Date: Mar. 25, 2009

Related U.S. Application Data
(60) Provisional application No. 60/847,988, filed on Sep. 29, 2006.
(60) Provisional application No. 60/847,987, filed on Sep. 29, 2006.
(60) Provisional application No. 60/928,652, filed on May 11, 2007.

(51) Int. Cl. A61M 21/02; A61M 16/10

(52) U.S. Cl. 600/27; 128/203.12; 128/203.26

(57) ABSTRACT

In one embodiment, an apparatus for ventilatory therapy (100) is provided, the apparatus comprising an apparatus body (122) defining a flow path for ventilatory gas, a diffuser (112) in operative communication with the flow path and at least one means for attaching the apparatus to ventialtory tubing or to a ventilatory device. In an additional embodiment, the method of providing an aromatherapy or aromatic substance to a subject is provided, the method comprising operatively connecting the apparatus to ventialtory tubing or a ventilatory device, providing an aromatic or aromatherapy substance to the diffuser, passing a ventialtory gas through the flow path, diffusing or evaporating a portion of the aromatic or aroma therapy substance into the ventialtory gas and providing the ventialtory gas to a subject.

### PRIORITY CLAIM

This application claims the benefit under 35 U.S.C. §119(e) of U.S. Provisional Application No. 60/847,987, filed Sep. 29, 2006, titled “APPARATUS FOR IN-LINE DIFFUSION OF VOLATILE SUBSTANCES IN VENTILATORY ASSISTANCE EQUIPMENT,” U.S. Provisional Application No. 60/847,988, filed Sep. 29, 2006, titled “METHOD FOR THE MANAGEMENT OF VENTILATORY THERAPY SIDE EFFECTS THROUGH IN-LINE DIFFUSION OF VOLATILE SUBSTANCES,” and U.S. Provisional Application No. 60/928,652, filed May 11, 2007, titled “APPARATUS FOR DIFFUSION OF AROMATIC SUBSTANCES IN VENTILATORY ASSISTANCE EQUIPMENT,” the entirety of the contents of each of which are hereby incorporated by this reference

### TECHNICAL FIELD

The present invention relates to ventilation systems. More specifically, the present invention relates to apparatus and methods for the introduction of aromatic compounds into the air stream of a ventilation system.

### BACKGROUND

Continuous Positive Airway Pressure (CPAP) treatment is a common ameliorative treatment for breathing disorders including Obstructive Sleep Apnea (OSA). OSA is a sleep disorder that affects an estimated 5% of the population in which the muscles which normally hold the airway open during sleep, relax and ultimately collapse, sealing the airway. The sleep pattern of an OSA sufferer is characterized by repeated sequence of snoring, breathing difficulty, lack of breathing, waking enough to resume breathing, and returning to sleep. Often the sufferer is not consciously aware of the sleep disorder. OSA patients usually experience symptoms characteristic of sleep deprivation such as severe drowsiness, irritability, functional and mental limitations, etc.

CPAP treatment provides pressurized air or other breathable gas to the entrance of subject's airways at a pressure elevated above atmospheric pressure. The pressurized air supplied to the subject effectively assists the muscles to keep the subject's airway open, eliminating typical OSA sleep pattern. Non-Invasive Positive Pressure Ventilation (NIPPV) is another form of treatment for breathing disorders which can involve a relatively higher pressure of gas being provided to a subject's nasal mask during the inspiratory phase or respiration and a relatively lower pressure or atmospheric pressure being provided in the subject's nasal mask during the expiratory phase of respiration.

Typically, the ventilatory assistance for CPAP or NIPPV treatment is delivered to a subject by way of a nasal mask. Alternatively, a mouth mask, full face mask, or nasal prongs may be used.

Ventilatory devices broadly comprise a ventilator body containing a continuous source of air or other breathable gas generally in the form of a blower driven by an electric motor. The electric motor is typically controlled by a servo-controller under the control of a micro-controller unit. A hospital or other piped gas supply may also be used. The ventilator body may be connected to ventilatory tubing, which in turn may be connected to a subject mask which typically incorporates, or has in close proximity, an exhaust to atmosphere for venting exhaled gasses. A ventilatory device may further comprise a humidifier for the addition of water vapor to the ventilatory gas.

### DISCLOSURE OF THE INVENTION

In one embodiment, an apparatus for ventilatory therapy is provided, the apparatus comprising: an apparatus body defining a flow path for ventilatory gas; a diffuser in operative communication with said flow path; and at least one means for attaching the apparatus to ventilatory tubing or a ventilatory device.

In an additional embodiment, a method of providing an aromatherapy or aromatic substance to a subject is provided, the method comprising: operatively connecting apparatus such as that which is described immediately above to ventilatory tubing or a ventilatory device; providing an aromatic or aromatherapy substance to the diffuser; passing a ventilatory gas through the flow path; diffusing or evaporating a portion of said aromatic or aroma therapy substance into said ventilatory gas; and providing the ventilatory gas to a subject.

In further embodiments methods of providing an aromatherapy or aromatic substance to a subject are provided, the methods comprising: placing a diffuser in close proximity to the air intake of a ventilatory device; providing an aromatic or aromatherapy substance to the diffuser; diffusing or evaporating a portion of said aromatic or aroma therapy substance into air being uptaken by said air intake; and providing said uptaken air to a subject.

### BRIEF DESCRIPTION OF THE DRAWINGS

FIG. 1 is an exploded view of one embodiment of a device for ventilatory therapy.

FIG. 2 is a side view of an assembled device depicted in FIG. 1.

FIG. 3 is cross section of the perspective view of FIG. 2 along line C.

FIGS. 4A-4D are schematic representations of non-limiting examples of how an apparatus may be operatively connected to ventilatory tubing or a ventilatory device.

FIG. 5 is a schematic view of one embodiment of a device for ventilatory therapy.

FIG. 6 shows the device of FIG. 5 operatively placed proximal to a ventilatory device.

### MODE(S) FOR CARRYING OUT THE INVENTION

CPAP and NIPPV treatment is known to have medical side effects such as dehydration of the airways and nasal passages which may lead to Rhinitis. To address these side effects, manufacturers often supply humidifiers (both passive and heated) which may be attached to the ventilatory device.

The present invention provides apparatus for providing an aromatic or aroma therapy substance to a subject via a ventilatory device. Examples of ventilatory device which may be used in combination with devices of the present invention include, but are not limited to, piston positive airway pressure, continuous positive airway pressure, automatic positive airway pressure, variable positive airway pressure, bi-level positive airway pressure, spontaneous time positive airway pressure, and non-invasive positive pressure ventilation ventilators.

It will be apparent to those of ordinary skill in the art that the embodiments described herein, while illustrative, are not intended to so limit the invention or the scope of the appended claims. Those of ordinary skill will understand that various combinations or modifications of the embodiments presented herein may be made without departing from the scope of the present invention.

One embodiment of an apparatus comprises a flow path for ventilatory gas defined by the body of the apparatus and at least one connector for connecting the apparatus to the body of a ventilatory device or to ventilatory tubing providing the ventilatory gas to a subject and a means for providing an aromatic or aroma therapy substance to ventilatory gas passing through the flow path.

A further embodiment of an apparatus comprises a flow path for ventilatory gas defined by passing through at least two connectors for connecting the apparatus to the body of a ventilatory device or to ventilatory tubing providing the ventilatory gas to a subject and a means for providing an aromatic or aroma therapy substance to ventilatory gas passing through the flow path.

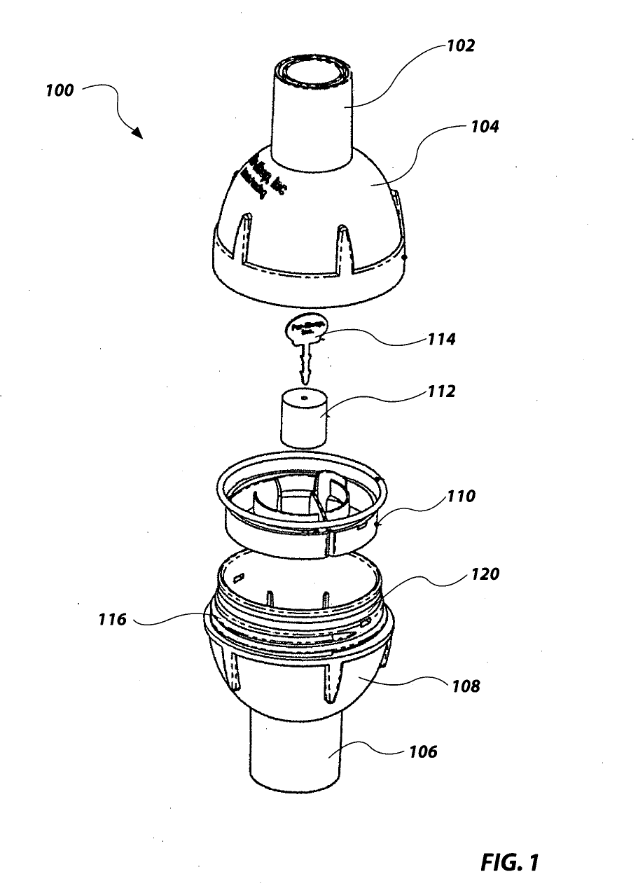

Referring now to drawing FIG. 1, therein is illustrated an exploded view of an apparatus generally at 100. As illustrated, apparatus 100 includes a first connector 102 attached to a first portion 104 of an apparatus body as well as a second connector 106 attached to a second portion 108 of an apparatus body. Further illustrated are support means 110, diffuser 112, and retention means 114. Also visible on second portion 108 of the apparatus body 108 are threads 116. Not visible are the mating counter-threads 118 on an interior surface of first portion 104 of the apparatus body 104. Further visible is optional gasket 120 or other sealing member.

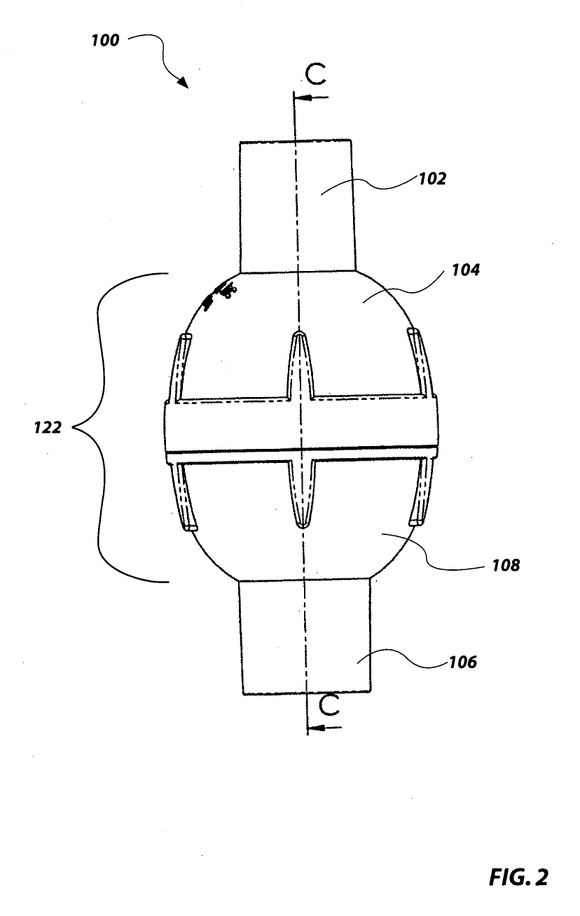

Referring now to drawing FIG. 2, therein is illustrated a perspective view of the assembled device depicted in FIG. 1. As illustrated, first portion 104 of the apparatus body and second portion 108 of the apparatus body may be joined to form the apparatus body 122.

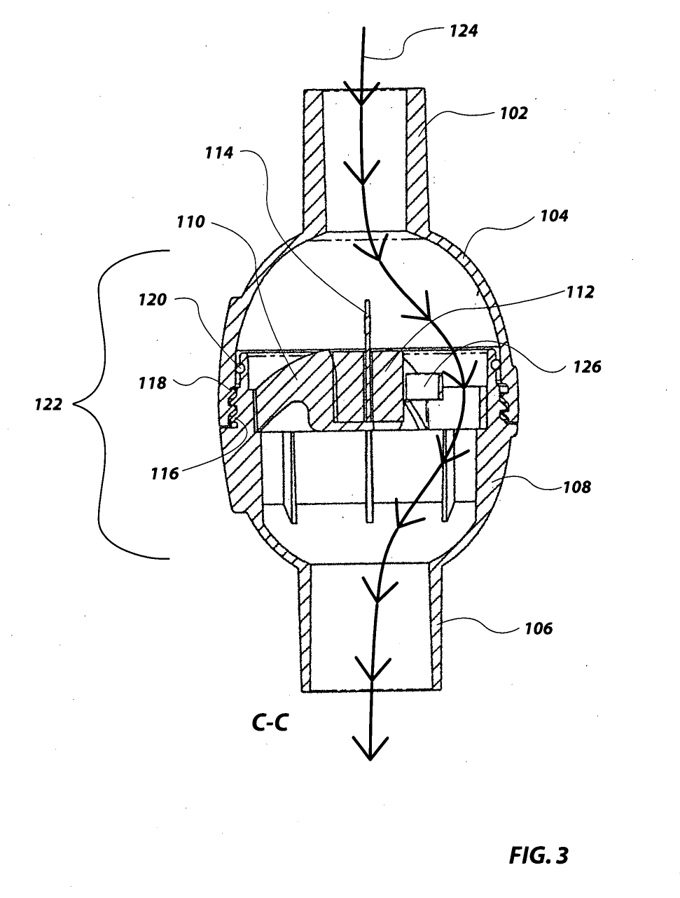

Referring now to drawing FIG. 3, therein is illustrated a cross section view of apparatus 100 along line C shown in FIG. 2. As illustrated therein, first portion 104 of the apparatus body and second portion 108 of the apparatus body may be joined to form apparatus body 122 via interlocking of threads 116 and counter-threads 118. Also visible is optional gasket 120 compressed between first portion 104 of the apparatus body and second portion 108 of the apparatus body. Further illustrated therein is flow path 124 for the flow of ventilatory gasses. Visible disposed within apparatus body 122 is support means 110 to which diffuser 112 is attached via retention means 114. Additionally illustrated in FIG. 3 is an optional heating element 126.

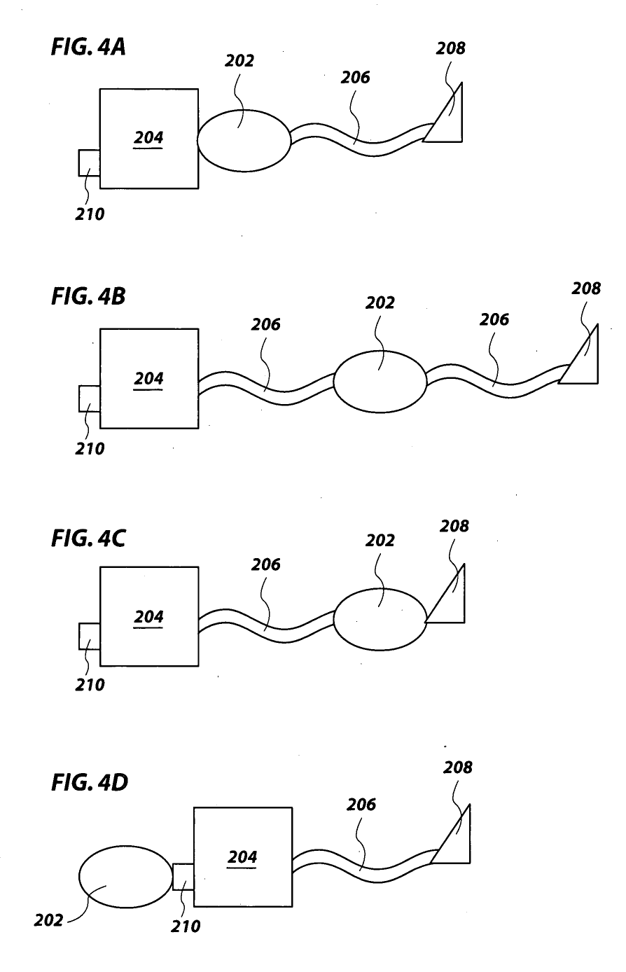

Referring now to FIGS. 4A-4D, therein are illustrated several non-limiting examples of configurations of an embodiment of an apparatus 202 operatively connected to a ventilatory device. As depicted therein, the apparatus 202 (which may include, for example, an apparatus such as has been described with respect to FIGS. 1 through 3) may be interposed between the ventilator body 204 and the ventilatory tubing 206 (FIG. 4A); between separate sections of ventilatory tubing 206 (FIG. 4B); between ventilatory tubing 206 and an interface device 208 (FIG. 4C); or attached to the air intake 210 of the ventilator body 204 (FIG. 4D).

In embodiments of the invention, interface device 208 may be any apparatus or means for delivering ventilatory gas to the respiratory system of a subject. Examples of interface devices 208 include, but are not limited to, masks, mouthpieces, and nasal tubing. In additional embodiments of the invention, the apparatus 202 may be in direct 20 contact with subject and/or may be the last portion of an overall ventilatory device through which ventilation gasses pass before moving into a subject.

In embodiments of the invention, a connector, such as connectors 102 and 106, may be any means for attaching to the apparatus to the body of a ventilatory device or to ventilatory tubing. Examples of such means include, but are not limited to, standard ventilation tubing connectors, connectors sized on configured for connection with ventilation tubing, compression fitting connectors, friction fitting connectors, screw type connectors, latching connectors, clips, hooks, hinges, etc. In embodiments of the invention, one or more of the connectors may be connected to or replaced by a patient interface device such as, but not limited to, a mask, mouthpiece, or nasal tubing. In particular embodiments, a connector may help define, form a portion of, or surround a portion of a flow path such as flow path 124.

As will be apparent to one of ordinary skill in the art, optional gasket 120 may be of any deformable material such that when first portion 104 of the apparatus body and second portion 104 of the apparatus body are joined, a relatively air tight seal is formed. As will be further apparent to one of ordinary skill in the art, first portion 104 of the apparatus body and second portion 108 of the apparatus body need not comprise separate, attachable, pieces. Thus, in embodiments of the invention, apparatus body 122 may be formed from a single piece. In additional embodiments of the invention, first portion 104 of the apparatus body, second portion 108 of the apparatus body, or apparatus body 122 may comprise an opening, door, or hatch or other means of providing access to diffuser 112.

As will be additionally apparent to one of ordinary skill in the art, in embodiments of the invention support means 110 need not comprise a separate piece, but may be formed directly into or integrated with another portion of the device. For example, support means 110 may be formed directly into or integrated with, but not limited to, first portion 104 of the apparatus body 104, second portion 108 of the apparatus body 108, or an apparatus body 122 that is formed as a unitary member.

In embodiments of the invention where apparatus body 122 is formed by the joining together of one or more pieces, such pieces may be permanently or removably joined using any means for joining. Examples of means for removably joining include, but are not limited to, threads (such as threads 116 and counter-threads 118), tape, clips, hooks, hinges, compression fittings, friction fittings, etc. Examples of means for permanently joining include, but are not limited to, soldering, melting, adhesives, pop-up clips, etc.

As will be apparent to one of ordinary skill of the art, support means 110 may be of any form or shape capable of supporting or holding in place diffuser 112. Examples of forms or shapes capable of supporting or holding in place diffuser 112 include, but are not limited to, cages, pockets, recesses, prongs, etc. In embodiments of the invention, one or more portions of the support means may pass through one or into one or more portions of diffuser 112. In additional embodiments, support means may support or hold in place diffuser 112 such that diffuser 112 is either removable or non-removable. Although depicted with retention means 114, retention means 114 is optional if the support means 110 adequately retains diffuser 112 without the need for additional retention (e.g., where likelihood of diffuser 112 separating from support means is unlikely without active intervention).

As will be additionally apparent to one of ordinary skill in the art, in embodiments of the invention comprising retention means 114, retention means 114 need not comprise a separate piece, but may be formed directly into or integrated with another portion of the device. For example, retention means 114 may be formed directly into or integrated with, but not limited to, first portion 104 of the apparatus body, second portion 108 of the apparatus body, support means 110, or an apparatus body 122 that is formed as a unitary member. Retention means 114 may be any means for retaining diffuser in place. Examples of a retention means 114 may include, but are not limited to, pins, doors, cages, clips, bars, bands, etc. In embodiments of the invention, one or more portions of the retention means 114 may pass through or into one or more portions of diffuser 112.

In embodiments of the invention, diffuser 112 may comprise any material or means capable of retaining and evaporating and/or diffusing an aromatic or aroma therapy substance: Examples of materials or means capable of retaining and evaporating and/or diffusing an aromatic or aroma therapy substance or means for providing an aromatic or aroma therapy substance to ventilatory gas include, but are not limited to atomizers, nebulizers, fiber pads, cloths, gels, absorbent material, and/or porous substances.

In embodiments of the invention, aromatic or aromatherapy substances may be any volatile substance which may be diffused or evaporated from a diffuser 112. Examples of aromatic or aromatherapy substances include, but are not limited to, volatile drugs, treatments, and/or essential oils such as peppermint, lemon, lavender, eucalyptus, etc.

In particular embodiments, optional heating element 126 may be any device or means for supplying heat to diffuser 112. Examples of devices or means for supplying heat to diffuser 112 include, but are not limited to, electric light bulbs, electric heating elements, and/or chemical heating elements. In further embodiments, optional heating elements 126 may be placed or positioned in operative proximity to the diffuser 112.

In embodiments of the invention, portions of the apparatus may be created or molded from any suitable substance. Non limiting examples of such substances include, but are not limited to plastics, composites, ceramics, and/or metals. In further embodiments of an apparatus, all or some portions of an apparatus are sterilizable and/or disposable. As used herein, sterilizable refers to the ability of an apparatus to undergo a standard sterilization procedure to completion and retain its form and ability perform the function it was designed for. Examples of standard sterilization procedures include, but are not limited to, radiation treatments, autoclaving, and treatment with biocidal substances. In further embodiments of the invention, the apparatus or portions of the apparatus may be impregnated and/or coated with substances that prevent the colonization of microorganism on or within the apparatus.

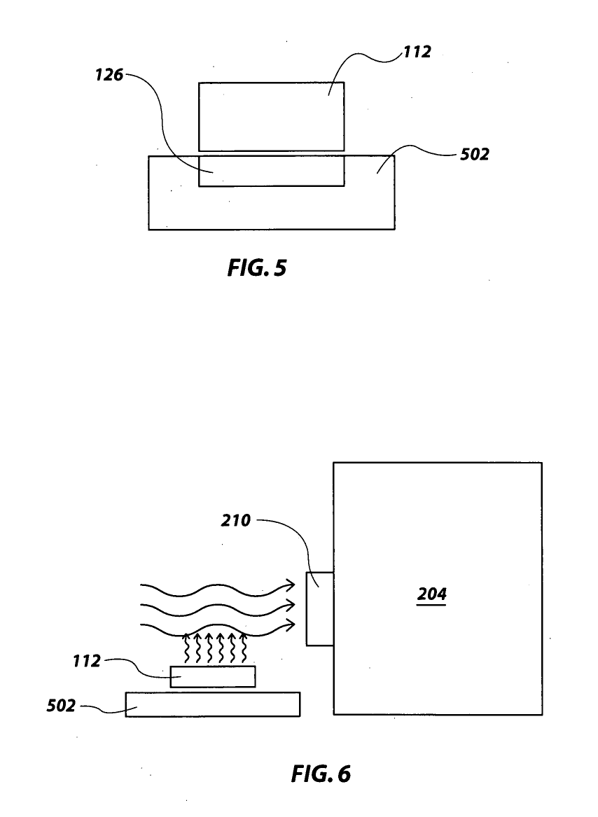

Referring now to drawing FIG. 5, illustrated therein is a further embodiment of an apparatus for providing an aromatic or aroma therapy substance to a subject via a ventilatory device. Illustrated therein is a plate 502, which may optionally comprise a heating element 126, on which is placed a diffuser 112. As will be apparent to one of ordinary skill in the art, plate 502 may be any vessel or object for supporting or retaining diffuser 112. Although not illustrated, plate 502 may comprise a means for aiding in the retention of diffuser 112. Examples of means for aiding in the retention of diffuser 112 include, but are not limited to, a depression, cage, pocket, recess, hook, and/or prong. Further embodiments of a plate 502 may comprise a means for aiding in the retention or support of a bottle or other vessel. In embodiments, such a bottle or other vessel may contain an aromatic or aromatherapy substance for use with diffuser 112. Means for aiding in the retention or support of a bottle or other vessel include, but are not limited to, a depression, cage, pocket, recess, hook, and/or prong.

Referring now to drawing FIG. 6, illustrated therein is the apparatus of FIG. 5 in use with a ventilatory device. As depicted therein, the apparatus of FIG. 5 may be placed next to or proximal to an air intake 210 of a ventilator body 204. The apparatus of FIG. 5 may be positioned such that aromatic or aroma therapy substances diffusing or evaporating from diffuser 112 are drawn into the air intake 210 of the ventilator body 204 along with incoming ventilatory gas.

Methods of providing an aromatic or aromatherapy substance to a subject via a ventilatory device are also disclosed. In one embodiment of a method, an apparatus as described supra is provided and an aromatic or aromatherapy substances is applied to a diffuser, such as diffuser 112. The apparatus may then be placed in the flow path of the ventilatory gas such that ventilatory gas proceeds through a flow path, such as flow path 124. Aromatic or aromatherapy substance may diffuse or evaporate from the diffuser and into the ventilatory gas and provided to the subject in conjunction with the ventilatory gas.

In embodiments of the invention, the ventilatory gas may be gas at any stage of a ventilatory process including, but not limited to, uptake or intake gas proceeding into the body of a ventilatory device, ventilatory gas within the body of a ventilatory device, and gas passing through or exiting ventilatory tubing en route to being provided to a subject.

In another embodiment of a method according to the present invention, an apparatus according to the present invention is provided and an aromatic or aromatherapy substances is applied to a diffuser 112. The device then may be placed in close proximity to the air intake 210 of a ventilator body 204. Aromatic or aromatherapy substance will diffuse or evaporate from diffuser 112 and into the gas entering the ventilator body via air intake 210 and be provided to the subject in conjunction with the ventilatory gas.

In a further embodiment of a method according to the present invention, aromatic or aromatherapy substances may be applied to a filter, air filter, or other fiber pad, cloth, gel, absorbent material, and/or porous substance that is associated with the normal function of a ventilatory device such that the aromatic or aromatherapy substance will diffuse or evaporate from a filter, air filter, or other fiber pad, cloth, gel, absorbent material, and/or porous substance that is associated with the normal function of a ventilatory device and into the ventilatory gas and be provided to the subject in conjunction with the ventilatory gas.

In yet another embodiment of a method according to the present invention, one or more of the methods disclosed herein may be used in the treatment of a condition associated with ventilator therapy. Examples of conditions associated with ventilator therapy include, but are not limited to, Rhinitis, nasal congestion, etc. In yet a further embodiment of a method according to the present invention, one or more of the methods disclosed herein may be used to increase subject compliance with ventilator therapy.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An apparatus for ventilatory therapy, the apparatus comprising:
an apparatus body defining a flow path for ventilatory gas;
a diffuser in operative communication with the flow path; and
at least one means for attaching the apparatus to ventilatory tubing or to a ventilatory device.

2. The apparatus according to claim 1, further comprising an aromatic or aromatherapy substance retained by the diffuser.

3. The apparatus according to claim 1, wherein the at least one means for attaching the apparatus to a ventilatory tubing or to a ventilatory device defines a portion of the flow path.

4. The apparatus according to claim 1, wherein the at least one means for attaching the apparatus to a ventilatory tubing or to a ventilatory device comprises at least two connectors.

5. The apparatus according to claim 4, wherein the at least two connectors define a portion of the flow path.

6. The apparatus according to claim 4, wherein at least one of the at least two connectors comprises a connector sized and configured for connection with ventilation tubing.

7. The apparatus according to claim 1, further comprising a means for supporting the diffuser in the flow path.

8. The apparatus according to claim 7, further comprising a means for retaining the diffuser.

9. The apparatus according to claim 1, further comprising a heating element in operative proximity to the diffuser.

10. The apparatus according to claim 1, wherein the apparatus is sterilizable.

11. The apparatus according to claim 1, wherein the diffuser is removably coupled with the apparatus body.

12. The apparatus according to claim 1, wherein the apparatus further comprises a port allowing access to the diffuser.

13. A method of providing an aromatherapy or aromatic substance to a subject, the method comprising:
operatively connecting an apparatus to ventilatory tubing or to a ventilatory device;
wherein the apparatus comprises:
an apparatus body defining a flow path for ventilatory gas;
a diffuser in operative communication with the flow path; and
at least one means for attaching the apparatus to ventilatory tubing or to a ventilatory device;
providing an aromatic or aromatherapy substance to the diffuser;
passing a ventilatory gas through the flow path;
diffusing or evaporating a portion of the aromatic or aroma therapy substance into the ventilatory gas; and
providing the ventilatory gas to a subject.

14. The method according to claim 13, wherein operatively connecting the apparatus to ventilatory tubing or to a ventilatory device comprises operatively connecting the apparatus of claim 1 to the ventilatory device body and ventilatory tubing.

15. The method according to claim 13, wherein operatively connecting the apparatus to ventilatory tubing or to a ventilatory device comprises operatively connecting the apparatus of claim 1 to at least two sections of ventilatory tubing.

16. The method according to claim 13, wherein operatively connecting the apparatus of claim 1 to ventilatory tubing or to a ventilatory device comprises operatively connecting the apparatus to an interface device and ventilatory tubing.

17. The method according to claim 13, wherein operatively connecting the apparatus of claim 1 to ventilatory tubing or to a ventilatory device comprises operatively connecting the apparatus to the air intake of the ventilatory device.

18. The method according to claim 13, the method further comprising heating the diffuser.

19. A method of providing an aromatherapy or aromatic substance to a subject, the method comprising:
placing a diffuser in close proximity to the air intake of a ventilatory device;
providing an aromatic or aromatherapy substance to the diffuser;
diffusing or evaporating a portion of the aromatic or aroma therapy substance into air being uptaken by the air intake; and
providing the uptaken air to a subject.

20. The method according to claim 19, the method further comprising heating the diffuser.
